(19) United States
(12) Patent Application Publication
(10) Pub. No.: US 2019/0008854 A1
(43) Pub. Date: Jan. 10, 2019

(54) DRUG COMPRISING ARIPIPRAZOLE AND CILOSTAZOL

(71) Applicant: OTSUKA PHARMACEUTICAL CO., LTD., Tokyo (JP)

(72) Inventors: Hwa Kyoung SHIN, Busan (KR); Byung Tae CHOI, Busan (KR); Ki Whan HONG, Busan (KR)

(73) Assignee: OTSUKA PHARMACEUTICAL CO., LTD., Tokyo (JP)

(21) Appl. No.: 16/064,786

(22) PCT Filed: Dec. 22, 2016
(86) PCT No.: PCT/JP2016/088554

§ 371 Date: Jun. 21, 2018

(30) Foreign Application Priority Data
Dec. 25, 2015 (JP) PCT/JP2015/006489

(51) Int. Cl. A61K 31/496; A61K 31/4709; A61P 25/28; A61P 25/24; A61P 25/00

(52) U.S. Cl. CPC A61K 31/496; A61K 31/4709; A61P 25/00; A61P 25/24; A61P 25/28

(57) ABSTRACT

The present invention enables treatment and/or prevention of dementia, cognitive impairment, and vascular depression by a combination of aripiprazole and cilostazol, or the like. The combination contains aripiprazole and cilostazol, and is used for the treatment and/or prevention of at least one member selected from the group consisting of dementia, cognitive impairment, and vascular depression.

### TECHNICAL FIELD

The present invention relates to a medicament of aripiprazole and cilostazol, and particularly relates to a combination of aripiprazole and cilostazol for use in the treatment and/or prevention of dementia, cognitive impairment, and/or vascular depression.

### BACKGROUND ART

With society rapidly aging, increasing attention has been drawn to dementia and cognitive impairment. Diseases caused by vascular disorders, such as cerebral stroke and cerebral infarction, due to the aging of blood vessels have been becoming more serious problems, especially among the elderly. It is known that even if adequate treatment is given to a patient with a disease caused by a vascular disorder and improvement is seen, the patient is likely to exhibit mental change or have potential risk of exhibiting mental change at a later stage. For example, vascular depression known to develop after treatment of vascular disorders is becoming an increasingly serious problem (see, for example, Patent Literature 1 and 2).

In the meantime, drugs having a high-level effect against platelet aggregation are known to be usable in the treatment and prevention of vascular disorders in expectation of their inhibitory effect on blood clots, such as cerebral infarction (see, for example, Patent Literature 2).

Aripiprazole, also expressed as 7-{4-[4-(2,3-dichlorophenyl)-1-piperazinyl]butoxy}-3,4-dihydro-2(1H)-quinolinone, is a carbostyril derivative and is known as an antipsychotic drug used in the treatment of mental disorders. Aripiprazole has also been studied for its applications in treating depression caused by vascular disorders (see, for example, Non-patent Literature 1).

### CITATION LIST

### Patent Literature

### [PTL 1]

U.S. Pat. No. 4,659,693

### [PTL 2]

JP2007-523121

### Non-Patent Literature

### [NPL 1]

Y. R. Kim et al., Behavioural Brain Research, Vol. 287, (2015) pp. 294-303.

### SUMMARY OF INVENTION

### Technical Problem

As noted above, diseases caused by vascular disorders are treated by administering an antithrombotic drug or the like. However, further studies are needed to elucidate the effect of such antithrombotic drugs on mental disorders, and how the drugs contribute to improved treatment.

A main object of the present invention is to enable treatment and/or prevention of dementia, cognitive impairment, and vascular depression by using a combination of aripiprazole, known as an antipsychotic drug, with cilostazol, or the like.

### Solution to Problem

The present inventors conducted extensive research to achieve the above object and found that a combination of aripiprazole with cilostazol (i.e., active ingredients) can produce a therapeutic and/or preventive effect on dementia, cognitive impairment, and vascular depression. The inventors further conducted research and completed the present invention.

The present invention was completed based on the findings.

Item 1. A combination comprising aripiprazole and cilostazol for use in the treatment and/or prevention of at least one member selected from the group consisting of dementia, cognitive impairment, and vascular depression.

Item 2. The combination according to Item 1, which is a combination drug comprising the aripiprazole and the cilostazol.

Item 3. The combination according to Item 1, separately comprising (A) a medicament containing the aripiprazole and (B) a medicament containing the cilostazol.

Item 4. The combination according to any one of Items 1 to 3, wherein the dementia, cognitive impairment, and vascular depression are caused by cerebral stroke.

Item 5. The combination according to any one of Items 1 to 4, wherein the dementia is at least one member selected from the group consisting of vascular dementia and senile dementia.

Item 6. The combination according to Item 5, wherein the vascular dementia is cerebrovascular dementia.

Item 7. The combination according to Item 5 or 6, wherein the vascular dementia is dementia induced after cerebral ischemia caused by cerebral stroke.

Item 8. The combination according to any one of Items 1 to 4, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric-shock-induced amnesia, memory impairment associated with depression or anxiety, cognitive impairment in Parkinson's disease, Down's syndrome, cerebral stroke, traumatic brain injury, Huntington's disease, and attention deficit disorder.

Item 9. The combination according to Item 8, wherein the vascular cognitive impairment is cerebrovascular cognitive impairment.

Item 10. The combination according to Item 8 or 9, wherein the vascular cognitive impairment is cognitive impairment induced after cerebral ischemia caused by cerebral stroke.

Item 11. The combination according to any one of Items 1 to 4, wherein the vascular depression is cerebrovascular depression.

Item 12. The combination according to any one of Items 1 to 4, and 11, wherein the vascular depression is depression induced by an age-related vascular disorder.

Item 13. The combination according to any one of Items 1 to 4, 11, and 12, wherein the vascular depression is depression induced after cerebral ischemia caused by cerebral stroke.

Item 14. The combination according to any one of Items 1 to 13, which is administered such that the daily dose of the aripiprazole is 0.01 to 300 mg/day.

Item 15. The combination according to any one of Items 1 to 14, which is administered such that the daily dose of the cilostazol is 1 to 300 mg/day.

Item 16. The combination according to any one of Items 1 to 15, which is administered such that the daily dose of the aripiprazole is 0.01 to 300 mg/day, and the daily dose of the cilostazol is 1 to 300 mg/day.

Item 17. The combination according to any one of Items 1 to 16, wherein the cilostazol is present in an amount of 0.1 to 100 parts by mass per part by mass of the aripiprazole in the combination, the combination being in the form of either a combination drug or medicament (A).

Item 18. A method for treating and/or preventing at least one member selected from the group consisting of dementia, cognitive impairment, and vascular depression in a patient in need of such a treatment and/or prevention, the method comprising administering a combination of aripiprazole and cilostazol to the patient.

Item 19. The method according to Item 18, wherein the aripiprazole and the cilostazol are administered sequentially or simultaneously.

Item 20. The method according to Item 18 or 19, wherein the dementia, cognitive impairment, and vascular depression are caused by cerebral stroke.

Item 21. The method according to any one of Items 18 to 20, wherein the dementia is at least one member selected from the group consisting of vascular dementia and senile dementia.

Item 22. The method according to Item 21, wherein the vascular dementia is cerebrovascular dementia.

Item 23. The method according to Item 21 or 22, wherein the vascular dementia is dementia induced after cerebral ischemia caused by cerebral stroke.

Item 24. The method according to any one of Items 18 to 20, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder.

Item 25. The method according to Item 24, wherein the vascular cognitive impairment is cerebrovascular cognitive impairment.

Item 26. The method according to Item 25, wherein the cerebrovascular cognitive impairment is cognitive impairment induced after cerebral ischemia caused by cerebral stroke.

Item 27. The method according to any one of Items 18 to 20, wherein the vascular depression is cerebrovascular depression.

Item 28. The method according to Item 18, 19, 20, or 27, wherein the vascular depression is depression induced by an age-related vascular disorder.

Item 29. The method according to Item 18, 19, 20, 27, or 28, wherein the vascular depression is depression induced after cerebral ischemia caused by cerebral stroke.

Item 30. The method according to any one of Items 18 to 29, wherein the daily dose of the aripiprazole is 0.01 to 300 mg/day.

Item 31. The method according to any one of Items 18 to 30, wherein the daily dose of the cilostazol is 1 to 300 mg/day.

Item 32. The method according to any one of Items 18 to 31, wherein the combination is administered such that the daily dose of the aripiprazole is 0.01 to 300 mg/day, and the daily dose of the cilostazol is 1 to 300 mg/day.

Item 33. A medicament comprising cilostazol for use in the treatment and/or prevention of at least one member selected from the group consisting of dementia, cognitive impairment, and vascular depression, wherein the cilostazol is administered to a patient being administered aripiprazole.

Item 34. A medicament comprising cilostazol for use in the treatment and/or prevention of at least one member selected from the group consisting of dementia, cognitive impairment, and vascular depression, wherein the cilostazol is administered simultaneously with aripiprazole to a patient in need of administration of aripiprazole.

Item 35. A medicament comprising aripiprazole for use in the treatment and/or prevention of at least one member selected from the group consisting of dementia, cognitive impairment, and vascular depression, wherein the aripiprazole is administered to a patient being administered cilostazol.

Item 36. A medicament comprising aripiprazole for use in the treatment and/or prevention of at least one member selected from the group consisting of dementia, cognitive impairment, and vascular depression, wherein the aripiprazole is administered simultaneously with cilostazol to a patient in need of administration of cilostazol.

Item 37. The medicament according to any one of Items 33 to 36, wherein the dementia, cognitive impairment, and vascular depression are caused by cerebral stroke.

Item 38. The medicament according to any one of Items 33 to 36, wherein the dementia is at least one member selected from the group consisting of vascular dementia, and senile dementia.

Item 39. The medicament according to Item 38, wherein the vascular dementia is cerebrovascular dementia.

Item 40. The medicament according to Item 38 or 39, wherein the vascular dementia is dementia induced after cerebral ischemia caused by cerebral stroke.

Item 41. The medicament according to any one of Items 33 to 37, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder.

Item 42. The medicament according to Item 41, wherein the vascular cognitive impairment is cerebrovascular cognitive impairment.

Item 43. The medicament according to Item 41 or 42, wherein the vascular cognitive impairment is cognitive impairment induced after cerebral ischemia caused by cerebral stroke.

Item 44. The medicament according to any one of Items 33 to 37, wherein the vascular depression is cerebrovascular depression.

Item 45. The medicament according to any one of Items 33 to 37, and 44, wherein the vascular depression is depression induced by an age-related vascular disorder.

Item 46. The medicament according to any one of Items 33 to 37, 44, and 45, wherein the vascular depression is depression induced after cerebral ischemia caused by cerebral stroke.

Item 47. The medicament comprising cilostazol according to any one of Items 33, and 37 to 46, which is administered to a patient who is being administered aripiprazole in an amount of 0.01 to 300 mg/day.

Item 48. The medicament comprising cilostazol according to any one of Items 34, and 37 to 46, which is administered to a patient in need of administration of aripiprazole in an amount of 0.01 to 300 mg/day.

Item 49. The medicament comprising cilostazol according to any one of Items 33 to 46, which is administered such that the daily dose of the cilostazol is 1 to 300 mg/day.

Item 50. The medicament comprising aripiprazole according to any one of Items 35, and 38 to 46, which is administered to a patient who is being administered cilostazol in an amount of 1 to 300 mg/day.

Item 51. The medicament comprising aripiprazole according to any one of Items 36 to 46, which is administered to a patient in need of administration of cilostazol in an amount of 1 to 300 mg/day.

Item 52. The medicament comprising aripiprazole according to any one of Items 35 to 46, 50, and 51, which is administered such that the daily dose of the aripiprazole is 0.01 to 300 mg/day.

Item 53. Use of a combination of aripiprazole and cilostazol in the treatment and/or prevention of at least one member selected from the group consisting of dementia, cognitive impairment, and vascular depression.

Item 54. The use according to Item 53, wherein the combination is a combination drug comprising the aripiprazole and the cilostazol.

Item 55. The use according to Item 53 or 54, wherein the dementia, cognitive impairment, and vascular depression are caused by cerebral stroke.

Item 56. The use according to Item 53, wherein the combination separately comprises (A) a medicament containing the aripiprazole and (B) a medicament containing the cilostazol.

Item 57. The use according to any one of Items 53 to 56, wherein the dementia is at least one member selected from the group consisting of vascular dementia, and senile dementia.

Item 58. The use according to Item 57, wherein the vascular dementia is cerebrovascular dementia.

Item 59. The use according to Item 57 or 58, wherein the vascular dementia is dementia induced after cerebral ischemia caused by cerebral stroke.

Item 60. The use according to any one of Items 53 to 55, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder.

Item 61. The use according to Item 60, wherein the vascular cognitive impairment is cerebrovascular cognitive impairment.

Item 62. The use according to Item 60 or 61, wherein the vascular cognitive impairment is cognitive impairment induced after cerebral ischemia caused by cerebral stroke.

Item 63. The use according to any one of Items 53 to 55, wherein the vascular depression is cerebrovascular depression.

Item 64. The use according to any one of Items 53 to 55, and 63, wherein the vascular depression is depression induced by an age-related vascular disorder.

Item 65. The use according to any one of Items 53 to 55, 63, and 64, wherein the vascular depression is depression induced after cerebral ischemia caused by cerebral stroke.

Item 66. The use according to any one of Items 53 to 65, wherein the daily dose of the aripiprazole is 0.01 to 300 mg/day.

Item 67. The use according to any one of Items 53 to 66, wherein the daily dose of the cilostazol is 1 to 300 mg/day.

Item 68. The use according to any one of Items 53 to 67, wherein the daily dose of the aripiprazole is 0.01 to 300 mg/day, and the daily dose of the cilostazol is 1 to 300 mg/day.

Item 69. Aripiprazole and cilostazol for use in the treatment and/or prevention of at least one member selected from the group consisting of dementia, cognitive impairment, and vascular depression.

Item 70. The aripiprazole and cilostazol according to Item 69, which are administered sequentially or simultaneously.

Item 71. The aripiprazole and cilostazol according to Item 69 or 70, wherein the dementia, cognitive impairment, and vascular depression are caused by cerebral stroke.

Item 72. The aripiprazole and cilostazol according to any one of Items 69 to 71, wherein the dementia is at least one member selected from the group consisting of vascular dementia, and senile dementia.

Item 73. The aripiprazole and cilostazol according to Item 72, wherein the vascular dementia is cerebrovascular dementia.

Item 74. The aripiprazole and cilostazol according to Item 72 or 73, wherein the vascular dementia is dementia induced after cerebral ischemia caused by cerebral stroke.

Item 75. The aripiprazole and cilostazol according to any one of Items 69 to 71, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder.

Item 76. The aripiprazole and cilostazol according to Item 75, wherein the vascular cognitive impairment is cerebrovascular cognitive impairment.

Item 77. The aripiprazole and cilostazol according to Item 75 or 76, wherein the vascular cognitive impairment is cognitive impairment induced after cerebral ischemia caused by cerebral stroke.

Item 78. The aripiprazole and cilostazol according to any one of Items 69 to 71, wherein the vascular depression is cerebrovascular depression.

Item 79. The aripiprazole and cilostazol according to any one of Items 69 to 71, and 78, wherein the vascular depression is depression induced by an age-related vascular disorder.

Item 80. The aripiprazole and cilostazol according to Item any one of Items 69 to 71, 78 and 79, wherein the vascular depression is depression induced after cerebral ischemia caused by cerebral stroke.

Item 81. The aripiprazole and cilostazol according to any one of Items 69 to 80, which are used such that the daily dose of the aripiprazole is 0.01 to 300 mg/day.

Item 82. The aripiprazole and cilostazol according to any one of Items 69 to 81, which are used such that the daily dose of the cilostazol is 1 to 300 mg/day.

Item 83. The aripiprazole and cilostazol according to any one of Items 69 to 82, which are used such that the daily dose of the aripiprazole is 0.01 to 300 mg/day and the daily dose of the cilostazol is 1 to 300 mg/day.

Item 84. Use of aripiprazole and cilostazol for producing a medicament for treating and/or preventing at least one member selected from the group consisting of dementia, cognitive impairment, and vascular depression.

Item 85. The use according to Item 84, wherein the medicament is a combination drug comprising the aripiprazole and the cilostazol.

Item 86. The use according to Item 84 or 85, wherein the medicament separately comprises (A) a medicament containing the aripiprazole and (B) a medicament containing the cilostazol.

Item 87. The use according to any one of Items 84 to 86, wherein the dementia, cognitive impairment, and vascular depression are caused by cerebral stroke.

Item 88. The use according to any one of Items 84 to 87, wherein the dementia is at least one member selected from the group consisting of vascular dementia, and senile dementia.

Item 89. The use according to Item 88, wherein the vascular dementia is cerebrovascular dementia.

Item 90. The use according to Item 88 or 89, wherein the vascular dementia is dementia induced after cerebral ischemia caused by cerebral stroke.

Item 91. The use according to any one of Items 84 to 87, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder.

Item 92. The use according to Item 91, wherein the vascular cognitive impairment is cerebrovascular cognitive impairment.

Item 93. The use according to Item 91 or 92, wherein the vascular cognitive impairment is cognitive impairment induced after cerebral ischemia caused by cerebral stroke.

Item 94. The use according to any one of Items 84 to 87, wherein the vascular depression is cerebrovascular depression.

Item 95. The use according to any one of Items 84 to 87, and 94, wherein the vascular depression is depression induced by an age-related vascular disorder.

Item 96. The use according to any one of Items 84 to 87, 94, and 95, wherein the vascular depression is depression induced after cerebral ischemia caused by cerebral stroke.

Item 97. The use according to any one of Items 84 to 96, wherein the daily dose of the aripiprazole is 0.01 to 300 mg/day.

Item 98. The use according to any one of Items 84 to 97, wherein the daily dose of the cilostazol is 1 to 300 mg/day.

Item 99. The use according to any one of Items 84 to 98, wherein the daily dose of the aripiprazole is 0.01 to 300 mg/day and the daily dose of the cilostazol is 1 to 300 mg/day.

Item 100. A commercial package comprising a description concerning the combination or the medicament according to the precedent Items,

wherein the description states that the combination or the medicament is usable or to be used in the treatment and/or prevention of at least one member selected from the group consisting of dementia, cognitive impairment, and vascular depression.

Item 101. A method comprising administering aripiprazole and cilostazol to a patient with at least one disease selected from the group consisting of dementia, cognitive impairment, and vascular depression,

wherein the aripiprazole and the cilostazol are administered as a single drug or individual drugs,
wherein the aripiprazole and the cilostazol in the form of individual drugs are administered simultaneously or separately with a time interval.

### Advantageous Effects of Invention

According to the present invention, aripiprazole, known as an antipsychotic drug, produces a remarkably excellent effect in the treatment and/or prevention of dementia, cognitive impairment, and vascular depression when used in combination with cilostazol, having a blood clot inhibition activity, whose applications to mental disorders have not been fully known.

### BRIEF DESCRIPTION OF DRAWINGS

FIG. 1 shows the results of tests using middle cerebral artery occlusion models.

FIG. 2 shows the results of quantitative evaluation of CREB phosphorylation in mouse brain tissues.

FIG. 3 shows the results of the measurement of Heme Oxygenase-1 expression levels.

FIG. 4 shows the results of a test using bilateral common carotid artery stenosis models.

### DESCRIPTION OF EMBODIMENTS

Aripiprazole, used in the present invention, is a carbostyril derivative, also expressed as 7-{4-[4-(2,3-dichlorophenyl)-1-piperazinyl]butoxy}-3,4-dihydro-2(1H)-quinolinone. Aripiprazole is also known as 7-[4-[4-(2,3-dichlorophenyl)-1-piperazinyl]butoxy]-3,4-dihydro carbostyril, Abilify, OPC-14597, OPC-31, or BMS-337039. Aripiprazole has activity as an agonist for the serotonin receptor and dopamine receptor, and acts as an agonist or partial agonist for the serotonin 5HT_{1A} receptor and the dopamine D₂ receptor. Aripiprazole is a dopamine-serotonin system stabilizer. The scope of the present invention encompasses the metabolites of aripiprazole. Dehydroaripiprazole is one of the metabolites of aripiprazole.

Cilostazol is also expressed as 6-[4-(1-cyclohexyl-1H-tetrazol-5-yl)butoxy]-3,4-dihydro carbostyril.

The diseases treated and/or prevented by the present invention is dementia, cognitive impairment, and vascular depression.

Examples of dementia, cognitive impairment, and vascular depression include disorders caused by cerebral stroke.

Examples of dementia include vascular dementia and senile dementia. Vascular dementia is induced particularly by a cerebrovascular disorder. Thus, vascular dementia also includes frontotemporal dementia, alcoholic dementia, and lacunar dementia. More specific examples of vascular dementia include dementia induced by a cerebrovascular disorder, and examples of cerebrovascular disorders include cerebral stroke (e.g., cerebral infarction, cerebral hemorrhage, and subarachnoid hemorrhage). Still more specific examples of vascular dementia include dementia induced after cerebral ischemia caused by cerebral stroke.

Examples of cognitive impairment include Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder. Examples of vascular cognitive impairment include cerebrovascular cognitive impairment, and examples of cerebrovascular cognitive impairment include cognitive impairment induced after cerebral ischemia caused by cerebral stroke (for example, cognitive impairment induced after cerebral ischemia caused by cerebral infarction).

Vascular cognitive impairment is induced by a vascular disorder. More specific examples of vascular cognitive impairment include cognitive impairment induced by a cerebrovascular disorder. Examples of cerebrovascular disorders include cerebral stroke (e.g., cerebral infarction, cerebral hemorrhage, and subarachnoid hemorrhage). Still more specific examples of vascular cognitive impairment include cognitive impairment induced after cerebral ischemia caused by cerebral stroke(for example, cognitive impairment induced after cerebral ischemia caused by cerebral infarction).

Vascular depression is induced after a vascular disorder. More specific examples of vascular dementia include depression induced by a cerebrovascular disorder. Examples of cerebrovascular disorders include cerebral stroke (e.g., cerebral infarction, cerebral hemorrhage, and subarachnoid hemorrhage). Examples of vascular depression further include depression induced by an age-related vascular disorder.

Examples of vascular depression include post-cerebral stroke depression, post-cerebral infarction depression, and senile depression. For the diagnosis and symptoms of post-cerebral stroke depression and vascular depression, for example, DSM-5 (Diagnostic and Statistical Manual of Mental Disorders, 5th edition), Biol Psychiatry (1998; 43:705-712), etc., may be referred to.

The combination according to the present invention is useful as a CREB (cAMP response element binding protein) phosphorylation stimulating agent, a pCREB (phosphated CREB) stimulating agent, or an HO-1 (heme oxygenase-1) stimulating agent. The combination according to the present invention is also expected to serve as a BDNF (brain-derived neurotrophic factor) stimulating agent, a GSK3/β (glycogen synthase kinase 3 beta) phosphorylation stimulating agent, or a pGSK3/β (phosphated GSK3/β) stimulating agent.

The effect attributed to the fact that CREB is phosphorylated to form phosphorylated CREB (pCREB) is not particularly limited. For example, because of its ability to promote the synthesis of Bcl-2 (B-cell lymphoma 2), COX-2 (cyclooxygenase-2), tyrosine hydroxylase, BDNF, which is a neurotrophic factor, and NGF (nerve growth factor), which is also a neurotrophic factor, pCREB is expected to bring improvement in learning ability or memory ability, anti-apoptotic action, increases in neurotransmitter production, or enhancement of neurogenesis. The effect brought about by the production of pCREB also includes the effect disclosed in the literature Carlos A. Saura (Rev. Neurosci., Vol. 22(2): 153-169, 2011). HO-1 is known as a cytoprotective protein that protects cells from damage caused by oxidative stress. Thus, HO-1 is also expected to have, for example, the effect disclosed in the following literature: Stephan W. Ryter, Physiol Rev 86: 583-650, 2006. In addition, given the promoting effect on the production of BDNF or phosphorylation of GSK3/β, HO-1 shows its promising applications in Alzheimer's dementia, depression, anxiety disorder, bipolar disorder, schizophrenia, autism spectrum disorder (developmental disorder), Parkinson's disease, tauopathy (tau protein abnormality), or other diseases.

The following describe an embodiment of the present invention in detail.

The combination according to the present invention comprises, as active ingredients, aripiprazole and cilostazol. The combination, for example, may be in the form of a combination drug comprising aripiprazole and cilostazol in a single medicament, or in the form of a combination separately comprising (A) a medicament containing aripiprazole and (B) a medicament containing cilostazol.

The combination is used in the form of a typical pharmaceutical preparation. Pharmaceutical preparations are prepared using diluents and excipients generally used in pharmaceuticals, such as fillers, extenders, binders, humectants, disintegrators, surfactants, and lubricants. For these preparations, various pharmaceutical forms can be selected in accordance with the therapeutic purposes, and typical examples include tablets, pills, powders, fluids, suspensions, emulsions, granules, capsules, suppositories and injectable drugs (e.g., fluids and suspensions).

When the combination comprises medicament (A) and medicament (B), the pharmaceutical forms of medicament (A) and medicament (B) are not particularly limited, and may be the same or different, as long as the medicaments are in the form listed above.

When the combination is formed into tablets, a wide variety of carriers conventionally known in this technical field can be used. Examples of such carriers include excipients such as lactose, sucrose, sodium chloride, glucose, urea, starch, calcium carbonate, kaolin, crystalline cellulose, and silicic acid; binders such as water, ethanol, propanol, simple syrup, glucose solution, starch solution, gelatin solution, carboxymethyl cellulose, shellac, methylcellulose, potassium phosphate, and polyvinylpyrrolidones; disintegrators such as dry starch, sodium alginate, agar powder, laminaran powder, sodium hydrogen carbonate, calcium carbonate, polyoxyethylene sorbitan fatty acid esters, sodium lauryl sulfate, stearic acid monoglyceride, starch, and lactose; disintegration inhibitors such as sucrose, stearin, cacao butter, and hydrogenated oils; absorption enhancers such as quaternary ammonium base and sodium lauryl sulfate; humectants such as glycerol and starch; adsorbents such as starch, lactose, kaolin, bentonite, and colloidal silica; and lubricants such as purified talc, stearate, boric acid powder, and polyethylene glycol. Tablets can also be formed as tablets with ordinary coatings, if necessary. Examples of such tablets include sugar-coated tablets, gelatin-coated tablets, enteric-coated tablets, film-coated tablets, double-layer tablets, and multilayer tablets.

When the combination is formed into pills, a wide variety of carriers conventionally known in this technical field can be used. Examples of usable carriers include excipients such as glucose, lactose, starch, cacao butter, hydrogenated vegetable oil, kaolin, and talc; binders such as powdered gum arabic, powdered tragacanth, gelatin, and ethanol; and disintegrators such as laminaran and agar.

When the combination is formed into suppositories, a wide variety of carriers conventionally known in this technical field can be used. Examples include polyethylene glycol, cacao butter, higher alcohols, esters of higher alcohols, gelatin, and semisynthetic glyceride.

Capsules are prepared according to an ordinary method typically by mixing an active ingredient compound with one or more of various carriers as listed above, and packing the mixture in hard gelatin capsules, soft capsules, or the like.

When the combination is prepared as an injectable drug, it is preferable that the fluid, emulsion, or suspension is sterilized, and is isotonic with blood. When the combination is formed into a fluid, emulsion, or suspension, any of diluents conventionally used in this technical field can be used. Examples of usable diluents include water, alcohols, such as methanol, ethanol, propanol, isopropanol, ethoxylated isostearyl alcohols, and polyoxylated isostearyl alcohols; macrogols; propylene glycols; polyoxyethylene sorbitan fatty acid esters; ketones, such as acetone; ethers, such as tetrahydrofuran; and dimethylformamide.

When prepared in the form of an injectable drug, the combination may contain salt, glucose, or glycerin in such a sufficient amount as to form an isotonic solution. A typical solubilizing agent, a buffer, or a soothing agent may be added. The combination may also optionally contain a coloring agent, a preserving agent, a flavoring, a sweetening agent as well as one or more other drugs.

When the combination is a combination drug, the aripiprazole content is not particularly limited, and can be suitably selected from a wide range. However, aripiprazole is preferably present in an amount of about 0.1 to 35% by mass in the combination drug. The cilostazol content is not particularly limited, and can suitably selected from a wide range. However, cilostazol is preferably present in an amount of about 0.1 to 35% by mass in the combination drug.

When the combination separately comprises medicament (A) and medicament (B), the aripiprazole content is not particularly limited, and can be suitably selected from a wide range. However, aripiprazole is typically present in an amount of about 0.1 to 70% by mass in medicament (A). The cilostazol content is not particularly limited, and can be suitably selected from a wide range. However, cilostazol is typically present in an amount of about 0.1 to 70% by mass in medicament (B).

The cilostazol content in the combination (i.e., a combination drug or medicament (B)) is preferably about 0.1 to 100 parts by mass per part by mass of aripiprazole present in the combination (i.e., a combination drug or medicament (A)).

The method for administering the combination is not particularly limited, and the combination is administered in accordance with the pharmaceutical form, the patient's age, the gender and other conditions, the extent of the disease, or the like. For example, the combination is administered orally, when in the form of tablets, pills, fluids, suspensions, emulsions, granules, or capsules. When in the form of an injectable drug, the combination is intravenously administered as a single drug or as a mixture containing a typical replacement fluid such as glucose and amino acids. The combination in the form of injectable drug is optionally administered as a single drug through an intramuscular, intracutaneous, subcutaneous, or intraperitoneal route. The combination is administered rectally when in the form of a suppository.

When the combination comprises medicament (A) and medicament (B), medicament (A) and medicament (B) may be administered sequentially or simultaneously.

When medicament (A) and medicament (B) are administered sequentially, medicament (A) may be administered first, and medicament (B) next, or vice versa. Specific examples include the method comprising administering cilostazol using medicament (B) to a patient already being administered aripiprazole using medicament (A) and the method comprising administering aripiprazole using medicament (A) to a patient already being administered cilostazol using medicament (B). The term “patient” as used herein include patients with diseases and/or disorders described above.

As used herein, “being administered aripiprazole” and “being administered cilostazol”, respectively, refer to the state before aripiprazole is substantially cleared from blood, and the state before cilostazol is substantially cleared from blood.

Specific examples of simultaneous administration of medicament (A) and medicament (B) includes a method comprising simultaneously administering (A) a medicament containing aripiprazole and (B) a medicament containing cilostazol to a patient in need of administration of aripiprazole using medicament (A) or a patient in need of administration of cilostazol using medicament (B). The patient in need of administration of aripiprazole or cilostazol is a patient with diseases and/or disorders described above.

The dose of the combination is suitably selected in accordance with the dose regimen, the patient's age, the gender and other conditions, the extent of the disease, or the like. However, the daily dose of aripiprazole is typically about 0.01 to 300 mg/day, preferably 0.1 to 100 mg/day, and more preferably 1 to 30 mg/day. The daily dose of cilostazol is typically about 1 to 300 mg/day, preferably 10 to 250 mg/day, and more preferably 50 to 200 mg/day.

### EXAMPLES

The following Examples and Comparative Examples describe the present invention in more detail. However, the present invention is not limited to the following embodiments.

### Example 1

### 1.1: In Vivo Test (Middle Cerebral Artery Occlusion Model)

In this experiment, 30 male 10-week-old C57BL/6J mice were divided into the following groups. To each group, 6 mice were allocated.

### Grouping

### 1) Control Group

### 2) Group Administered Vehicle

### 3) Group Administered Cilostazol Alone

### 4) Group Administered Aripiprazole Alone

### 5) Group Administered Cilostazol and Aripiprazole

A 20% DMSO aqueous solution served as a vehicle. The dose of cilostazol and the dose of aripiprazole were each 3 mg/kg. The dose of cilostazol and the dose of aripiprazole for the group administered cilostazol and aripiprazole were also 3 mg/kg each. The medicaments were each dissolved in a 20% DMSO aqueous solution and administered to the mice.

### Test Schedule

All of the mice were given a 14-day habituation period. On the day following the final day of the habituation period, the 4 mouse groups other than the control group underwent surgery, described later, to induce middle cerebral artery occlusion. From day 2 after the surgery, the mice of each group were orally administered their medicament once a day over 16 consecutive days as determined for each group described above and also given chronic mild stress described later. At the points 4 weeks, 5 weeks and 6 weeks after the middle cerebral artery occlusion surgery, all of the mice were subjected to the open field test, sucrose preference test (sucrose consumption test), and forced swimming test, which are described later. At the point 6 weeks after the middle cerebral artery occlusion surgery, all of the mice were subjected to the Morris water maze test. After the completion of the Morris water maze test, the brains of all of the mice were collected to prepare immunostaining samples of the brain tissues, and the state of the brain tissues were evaluated.

### 1.2: Middle Cerebral Artery Occlusion Surgery

Male C57BL/6J mice underwent surgery to induce middle cerebral artery occlusion (“MCAO”) under anesthesia with 1.5% isoflurane (in 80% N₂O and 20% O₂). The depth of anesthesia was checked by the absence of cardiovascular changes in response to tail pinch. The rectal temperature was kept at 36.5-37.5° C. using a thermostatically controlled heating mat (Panlab, Harvard Apparatus) during the surgery. A silicone-coated monofilament was introduced into the internal carotid artery and allowed to advance so as to occlude the internal carotid artery. The monofilament was withdrawn from the internal carotid artery 30 minutes after occlusion. The cerebral blood flow of all of the mice subjected to the surgery was measured to confirm the achievement of consistent and similar levels of ischemic induction. To measure the cerebral blood flow, a laser Doppler (PeriFlux Laser Doppler System 5000; Perimed, Stockholm, Sweden) with a flexible probe was used, and the obtained data were continuously recorded using a data acquisition and analysis system (PowerLab, AD Instruments, Medford, Mass.) and stored in a computer.

### 1.3: Chronic Mild Stress (CMS)

Chronic mild stress was caused by randomly adding the following 7 different stresses for 16 consecutive days.

1) Food and water deprivation for 20 hours
2) Water deprivation for 18 hours
3) 45° cage tilt for 17 hours
4) Overnight illumination (placed under illumination for a total of 36 consecutive hours)
5) Soiled cage (200 ml of water was added to 100 g of sawdust, and cages were bedded with the water-containing sawdust; the mice were placed in the cages for 21 hours)
6) Forced swimming in 4° C. water (5 minutes)
7) Pairs of mice were each placed in one cage (bottom surface: 28 cm×42 cm, height: 20 cm) for 2 hours.

### 1.4: Open Field Test

The mice were placed at the center of cages made from white polyethylene (30-cm-square, height: 40 cm). The distance over which the mice traveled in a predetermined time was monitored with a video-tracking system using the Smart software (Panlab, Barcelona, Spain).

### 1.5: Sucrose Preference Test

After food and water deprivation for 20 hours, a sucrose preference test was performed. Two bottles, one containing a 1% sucrose aqueous solution and the other containing water, were set in each cage, and the mice were placed in the cages for 1 hour, followed by measurement of the amount drank by the mice for both bottles. At the point 30 minutes after the start of the 1-hour test, the two bottles were weighed, and the position of the two bottles was changed, followed by continuation of the test for the remaining 30 minutes. The baseline value for the sucrose preference test was determined by measuring the value before starting to add the chronic mild stress. The sucrose preference was calculated according to the following equation:

sucrose aqueous solution intake (g)+water intake (g).

### 1.6: Forced Swim Test

Antidepressant-like activities were evaluated by performing the forced swim test. One day before the test, mice were forced to swim in a glass cylinder (height: 15 cm, diameter: 10 cm) containing 25° C. water for 15 minutes. On the test day, the mice were forced to swim in the water-containing glass cylinder for 5 minutes in the same manner, and the behaviors of the mice were recorded with a digital camera (E8400, Nikon Corporation, Japan), and the immobile time was measured.

### 1.7: Morris Water Maze Test

Spatial learning and memory decline were evaluated by performing the Morris water maze test. The Morris water maze test was performed in accordance with the procedure of Takeda S. et al. (Brain Res., (2009), Vol. 1280, 137-147). The maze consisted of a 1.15-m-diameter circular pool painted flat white, and was provided with a 10-cm-diameter circular platform halfway between the center of the pool and the edge. The platform was set 1 cm below the surface of the water. The water temperature of the circular pool was maintained at 19-21° C. The circular pool was located in a test room that contains many cues external to the maze. The position of the cues remained unchanged throughout the water maze test. Between a series of test trials, the mice were randomly placed in one of four directional starting locations (north, south, east, and west) in the pool facing the wall of the circular pool, and the test was started. The mice were given a maximum of 180 seconds to reach the platform submerged in water. When a mouse was unable to reach the platform within 180 seconds, the mouse was guided to the platform and placed on the platform for 10 seconds. Swimming was video-recorded, and the escape latency to reach the platform was analyzed using the Smart software (Panlab, Barcelona, Spain). The mean of latency time from the platform was analyzed.

### 1.8: Immunofluorescence

Mice received intracardial perfusion with PBS followed by fixative containing 4% paraformaldehyde under chloral hydrate anesthesia. Thereafter, the brains were removed from the mice and fixed by immersion in 4% paraformaldehyde for 24 hours. The fixed brains were immersed in a 30% sucrose solution for 48 hours at 4° C. The obtained brains were frozen and 30-μm-thick sections were obtained with a cryostat. The obtained sections were incubated overnight with the following primary antibodies in an antibody dilution buffer (PBS containing 1% of BSA and 0.3% of triton X-100) at 4° C.: the primary antibodies are phospho-CREB (pCREB, Ser133, Santa Cruz, Calif., USA), neuronal nuclei (NeuN, Millipore Corporation), and tyrosine hydroxylase (TH, Millipore Corporation). After being washed with PBS, the sections were incubated with a fluorescent secondary antibody (Vector Laboratories, Inc., Burlingame, Calif., USA) and DAPI (Invitrogen Corporation, Carlsbad, Calif., USA) for 2 hours and 30 minutes in a darkroom. Images were captured with a fluorescence microscope (Carl Zeiss, Inc., Goettingen, Germany).

### 1.9: Statistical Analysis

All of the data were expressed as the mean±standard error of the mean. The statistical analysis was performed using ANOVA. When a statistically significant effect is found, post hoc analysis is performed to detect the difference between the groups. A value of P<0.05 was accepted as being statistically significant.

### 1.10: Test Results

1.10.1

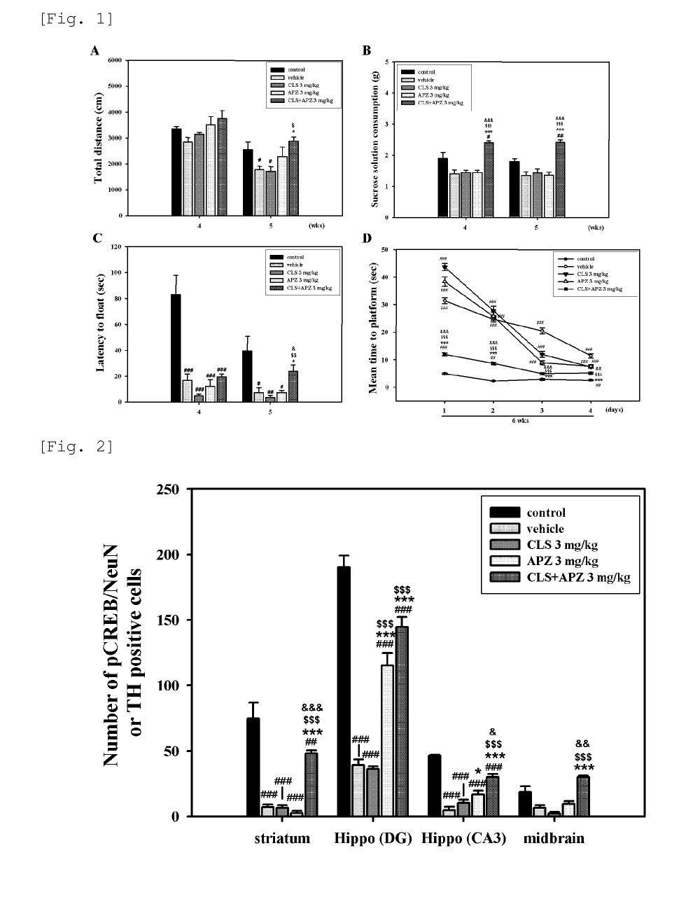

FIG. 1 shows the results of the open field test, sucrose preference test, forced swimming test, and Morris water maze test. In FIG. 1, graphs A, B, C, and D respectively show the results of the open field test, the results of the sucrose preference test, the results of the forced swimming test, and the results of the Morris water maze test. ^{#}P<0.05, ^{##}P<0.01, and ^{###}P<0.001 versus the control group. *P<0.05, **P<0.01, and ***P<0.001 versus the group administered vehicle. ^{$}P<0.05, ^{$$}P<0.01, and ^{$$$}P<0.001 versus the group administered cilostazol alone. ^{&}P<0.05, ^{&&}P<0.01, and ^{&&&}P<0.001 versus the group administered aripiprazole alone.

In the open field test, the total distance traveled by the mice in the group administered vehicle was significantly shorter than that of the mice in the control group. However, the total distance traveled by the mice in the group administered cilostazol and aripiprazole was significantly longer than that of the mice in the group administered vehicle.

In the sucrose preference test, the mice in the group administered cilostazol and aripiprazole exhibited a significantly higher intake of the sucrose aqueous solution than the mice in the group administered vehicle, the mice in the group administered cilostazol alone, or the mice in the group administered aripiprazole alone.

In the forced swimming test, the mice in the group administered vehicle exhibited a significantly shortened immobile time than the mice in the control group. However, the mice in the group administered cilostazol and aripiprazole exhibited a significantly extended immobile time than the mice in the group administered vehicle.

In the Morris water maze test, the mice in the group administered vehicle exhibited significantly longer escape latency than the mice in the control group. However, the mice in the group administered cilostazol and aripiprazole exhibited significantly shorter escape latency than the mice in the group administered vehicle, the mice in the group administered cilostazol alone, or the mice in the group administered aripiprazole alone.

1.10.2

FIG. 2 shows the results of quantified immunostaining analysis. The vertical axis indicates the sum of the number of cells positive for both pCREB and NeuN and the number of cells positive for both pCREB and TH. In FIG. 2, ^{#}P<0.01 and ^{##}P<0.001 versus the control group, *P<0.05, **P<0.01, and ***P<0.001 versus the group administered vehicle, ^{$$}P<0.01 and ^{$$$}P<0.001 versus the group administered cilostazol alone, and ^{&}P<0.05, ^{&&}P<0.01, and ^{&&&}P<0.001 versus the group administered aripiprazole alone.

The mice in the group administered vehicle exhibited a significantly lower sum of the number of cells positive for both pCREB and NeuN and the number of cells positive for both pCREB and TH in the brain tissues of any of the striatum, hippocampus, or midbrain than the mice in the control group. However, the mice in the group administered cilostazol and aripiprazole exhibited a significantly higher sum of the number of cells positive for both pCREB and NeuN and the number of cells positive for both pCREB and TH in the brain tissues than the mice in the group administered vehicle, the mice in the group administered cilostazol alone, or the mice in the group administered aripiprazole alone.

### Example 2

### 2.1: In Vitro Test

The effect of aripiprazole and/or cilostazol on neuronal cells was evaluated. Specifically, the expression level of heme oxygenase-1 (“HO-1”) was measured using Western blotting. HO-1 is a neuroprotective protein that protects cells from damage caused by oxidative stress.

HT22 neuronal cells (mouse hippocampus-derived neuronal cell line) were cultured in a Dulbecco's modified Eagle's medium (DMEM) containing 10% fetal bovine serum, 100 units/mL of penicillin, and 100 μg/mL of streptomycin. Thereafter, the cultured cells were divided into the following 4 groups: [1] a non-treated group, [2] a group treated with aripiprazole (1 μM), [3] a group treated with cilostazol (1 μM), and [4] a group treated with aripiprazole (1 μM)+cilostazol (1 μM), and the divided cells were subjected to respective treatments. Each treatment was performed by exposing the cell groups to respective substances for 6 hours. Thereafter, the treated cells were collected and lysed in a lysis buffer to prepare samples for electrophoresis, followed by polyacrylamide gel electrophoresis. The proteins separated by polyacrylamide gel electrophoresis were transferred onto PVDF membranes, and the PVDF membranes were treated with blocking buffer. The PVDF membranes were then sequentially treated with the primary antibody of HO-1 (Assay Design), the secondary antibody, and a chemiluminescent reagent in the Supersignal West Dura Extended Duration Substrate Kit (Pierce Chemical). Subsequently, the signals from bands on the PVDF membranes were measured with a detector. The expression level of HO-1 protein was normalized to β-actin level for evaluation.

### 2.2: Statistical Analysis

The results were expressed as the mean±standard error of the mean. The statistical analysis was performed using ANOVA. In the confirmation of statistically significant differences, Bonferroni's multiple comparison test was performed as a post hoc analysis to detect the differences between groups.

### 2.3: Test Results

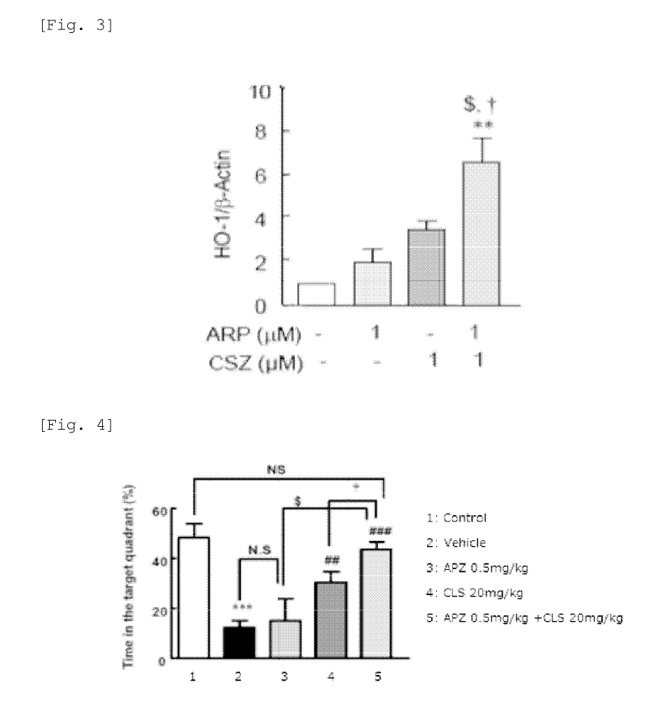

FIG. 3 shows the results of measurement of the HO-1 protein expression levels. In FIG. 3, **P<0.01 versus the non-treated sample, ^{†}P<0.05 versus the sample treated with aripiprazole (ARP 1 μM), and ^{$}P<0.05 versus the sample treated with cilostazol (CSZ 1 μM).

The HT22 neuronal cells treated with both aripiprazole and cilostazol exhibited a significantly higher HO-1 expression level than the non-treated HT22 neuronal cells, the HT22 neuronal cells treated with aripiprazole alone, or the HT22 neuronal cells treated with cilostazol alone.

### Example 3

### 3.1: In Vivo Test (Bilateral Common Carotid Artery Stenosis Model)

Mouse models with bilateral common carotid artery stenosis were prepared, and the Morris water maze test was performed on the mice models to evaluate the spatial learning and memory abilities. Forty male 10-week-old C57BL/6J mice (purchased from Koatech, Seoul, Korea) were divided into the following groups for use. To each group, 8 mice were allocated.

### Grouping

### 1) Control Group

### 2) Group Administered Vehicle

### 3) Group Administered Aripiprazole Alone

### 4) Group Administered Cilostazol Alone

### 5) Group Administered Cilostazol and Aripiprazole

A 25% DMSO aqueous solution served as a vehicle. For the group administered cilostazol and aripiprazole as well, the dose of aripiprazole was 0.5 mg/kg and the dose of cilostazol was 20 mg/kg. Each medicament was dissolved in a 25% DMSO aqueous solution and orally administered to the mice.

### Test Schedule

All of the mice were given a 5-day habituation period. On the day following the final day of the habituation period, the mice of each group underwent surgery to induce stenosis in the bilateral common carotid arteries. From day 2 to day 29 after surgery, the mice of each group were orally administered their medicament as determined for each group described above. The Morris water maze test was performed 3 weeks after the stenosis surgery.

### 3.2: Bilateral Common Carotid Artery Stenosis Surgery

The mice underwent surgery to induce bilateral common carotid artery stenosis (“BCCAS”) in accordance with the procedure disclosed in Shibata M. et al. (Stroke, (2004), Vol. 35, 2598-2603). A midline neck incision was made to the mice to expose the bilateral common carotid arteries, and the arteries were separated from the surrounding tissues. A microcoil with an inner diameter of 0.18 mm was attached to the outer side of the separated bilateral common carotid arteries. The control group also underwent the same BCCAS surgery except that a microcoil was not attached.

### 3.3: A Spatial and Memory Ability Test

To the purpose for assessment of the above, the Morris water maze test was performed in accordance with the procedure disclosed in Park SH. et al. (Biochem. Biophys. Res. Com., (2011), Vol. 408, 602-608). The maze consisted of a 1.15-m-diameter circular pool painted flat white, and a sufficient amount of water filled the circular pool. Of the 4 quadrants constituting the circular pool, one quadrant was provided with a 10-cm-diameter circular platform. The platform was set 1 cm below the surface of the water in the circular pool. To make the platform invisible under the water surface, powdered milk was dissolved in the water filling the circular pool. The water temperature was maintained at 19-21° C. In the preliminary training, the time a mouse spent from finding the platform until standing up on the platform with four limbs touched on the platform was determined to be the escape latency. The training continued for 5 consecutive days, during which the platform remained in the same location. The mice were given a maximum of 90 seconds to reach the platform submerged in water. When a mouse was unable to reach the platform within 90 seconds, the mouse was guided to the platform, where the mouse stayed for 10 seconds, and was returned to the cage. In this case, the escape latency was recorded as 90 seconds.

Before BCCAS surgery, the mice in each group were subjected to training as listed below.

Day 1: the mice were allowed to swim freely for 90 seconds in the circular pool without the platform being set.
Day 2: the platform was set inside the circular pool, and the mice were trained to find the platform one time.
Day 3: the mice were trained to find the platform twice.
Day 4: the mice were trained to find the platform three times.

A spatial and memory ability test was performed 3 weeks after the BCCAS surgery. The mice were allowed to swim freely in the circular pool with no platform. The swimming was video-recoded, and analyzed with the Smart software (Panlab, Barcelona, Spain). The ratio (%) of the time during which the mice spent within the quadrant where the platform was placed to the time during which the mice stayed in the circular pool in the training was calculated.

### 3.4: Statistical Analysis

All of the data were expressed as the mean±standard error of the mean. Student's t-test was used to determine significant differences. The effects of Aripiprazole alone, Cilostazol alone and their combination on the escape latency and on the time spent in the target quadrant in the Morris water maze test were analyzed with repeated ANOVA followed by the post hog Bonferroni's multiple comparison tests. A value of P<0.05 was accepted as being statistically significant.

### 3.5: Test Results

FIG. 4 shows the results of the spatial and memory ability test. In FIG. 4, ***P<0.01 versus the control group, ^{##}P<0.01 versus the group administered vehicle, ^{###}P<0.001 versus the group administered vehicle. ^{$}P<0.05, the group administered aripiprazole alone versus the group administered cilostazol and aripiprazole, ^{†}P<0.05, the group administered cilostazol alone versus the group administered aripiprazole and cilostazol.

The mice in the group administered vehicle spent less time within the quadrant where the platform was set during the training than the mice in the control group, exhibiting lowered spatial learning ability and memory ability. In contrast, the mice in the group administered cilostazol and aripiprazole spent significantly longer time within the quadrant than the mice in the group administered vehicle. In addition, the mice in the group administered cilostazol and aripiprazole spent significantly longer time within the quadrant than the mice in the group administered aripiprazole alone or cilostazol alone. The results reveal that the combinational effect demonstrated in Example 1 is also brought about in different test models.

1. A combination comprising aripiprazole and cilostazol for use in the treatment and/or prevention of at least one member selected from the group consisting of dementia, cognitive impairment, and vascular depression.

2. The combination according to claim 1, which is a combination drug comprising the aripiprazole and the cilostazol.

3. The combination according to claim 1, separately comprising (A) a medicament containing the aripiprazole and (B) a medicament containing the cilostazol.

4. The combination according to claim 1, wherein the dementia, cognitive impairment, and vascular depression are caused by cerebral stroke.

5. The combination according to claim 1, wherein the dementia, the cognitive impairment, and the vascular depression are associated with aging.

6. The combination according to claim 1, wherein the dementia is at least one member selected from the group consisting of vascular dementia and senile dementia.

7. The combination according to claim 1, wherein the cognitive impairment is at least one member selected from the group consisting of Alzheimer's disease, learning disabilities caused by degenerative disorders, declines in learning ability, memory or cognitive dysfunction such as mild cognitive impairment, senile cognitive impairment, age-related cognitive decline, cerebral senility, vascular cognitive impairment, AIDS-associated dementia, electric shock induced amnesia, memory impairment associated with depression or anxiety, cognitive impairment in Parkinson's disease, Down's syndrome, cerebral stroke, traumatic brain injury, Huntington's disease, and attention deficit disorder.

8. The combination according to claim 1, wherein the daily dose of the aripiprazole is 0.01 to 300 mg/day, and the daily dose of the cilostazol is 1 to 300 mg/day.

9. A medicament comprising cilostazol for use in the treatment and/or prevention of at least one member selected from the group consisting of dementia, cognitive impairment, and vascular depression, wherein the medicament comprising cilostazol is administered to a patient being administered aripiprazole or a patient in need of administration of aripiprazole.

10. A medicament comprising aripiprazole for use in the treatment and/or prevention of at least one member selected from the group consisting of dementia, cognitive impairment, and vascular depression, wherein the medicament comprising aripiprazole is administered to a patient being administered cilostazol or a patient in need of administration of cilostazol.

11. A commercial package comprising a description concerning the combination according to claim 1,
wherein the description states that the combination is usable or to be used in the treatment and/or prevention of at least one member selected from the group consisting of dementia, cognitive impairment, and vascular depression.

12. A method for treating and/or preventing at least one member selected from the group consisting of dementia, cognitive impairment, and vascular depression in a patient in need of such a treatment and/or prevention, the method comprising administering a combination of aripiprazole and cilostazol to the patient.

13. The method according to claim 12, wherein the aripiprazole and the cilostazol are administered sequentially or simultaneously.

14. A method comprising administering aripiprazole and cilostazol to a patient with at least one disease selected from the group consisting of dementia, cognitive impairment, and vascular depression,
wherein the aripiprazole and the cilostazol are administered as a single drug or individual drugs,
wherein the aripiprazole and the cilostazol in the form of individual drugs are administered simultaneously or separately with a time interval.

15. Use of a combination of aripiprazole and cilostazol in the treatment and/or prevention of at least one member selected from the group consisting of dementia, cognitive impairment, and vascular depression.

16. The use according to claim 15, wherein the combination is a combination drug comprising the aripiprazole and the cilostazol.

17. The use according to claim 15, wherein the combination separately comprises (A) a medicament containing the aripiprazole and (B) a medicament containing the cilostazol.

18. Use of aripiprazole and cilostazol for producing a medicament for treating and/or preventing at least one member selected from the group consisting of dementia, cognitive impairment, and vascular depression.
